(19) United States
(12) Patent Application Publication
(10) Pub. No.: US 2002/0094348 A1
(43) Pub. Date: Jul. 18, 2002

(54) Healing rock

(76) Inventor: Djavad Fouladi, Plantation, FL (US)

Correspondence Address: DJAVAD FOULADI, 1765 NW 72 Ave., Plantation, FL, 33313-4432, US

(21) Appl. No.: 09/759,886

(22) Filed: Jan. 16, 2001

(51) Int. Cl. A61K 33/00

(52) U.S. Cl. 424/600

(57) ABSTRACT

Discovery of a natural remedy contained inside the Healing Rock that is effective in healing the canker sores. Followed by invention of a natural oral mouthwash composition for treating canker sores, wherein composition consists of a blend of some small pieces (about half ounce) of said Healing rocks that are dissolved inside a cup of hot water. The patients who want to cure their canker sores can gargle the chilled composition for certain duration. Said Healing Rock is only a trademark name selected for a kind of natural rock, which exists in the Mother Nature that has never been tried before for purpose of curing the canker sores.

### FIELD OF DISCOVERY/INVENTION

The present discovery relates to a natural remedy contained in a kind of rock, which exists in the Mother Nature that is effective in healing the canker sores. The present discovery followed by an invention relates to natural oral mouthwash for treating canker sores.

### BACKGROUND OF THE DISCOVERY/INVENTION

About Canker Sores

Canker sores or aphthous ulcerations are painful sores that appear as small red dots that later turn to whiten shallow ulcers inside the membrane of the mouth. It will take a few days or week till it disappears. The cause of the canker sores is still unknown. Except pain reliever, There is no medication has ever been found to cure and prevent the canker sores.

About Me

I am 45 years old. I have experienced the discomfort of canker sores for the past decade. I had tried many different medications for treating my painful canker sores, but non-of them worked out well until late November 2000 when I accidentally tried the Healing Rock. It truly worked for me by actively curing my canker sores. Fortunately I have found the solution to my health problem that can benefit several thousands of people who have the same problem.

### SUMMARY OF THE DISCOVERY/INVENTION

Discovery of power of Healing Rock for treating canker sores. Its ability may also work preventing other gum diseases. Such as gingivitis, bad breath, dental plaque, tooth decay, cold sores, warts, fever blisters, as well as the elimination, or reduction of oral microflora, bacteria, or other oral viral responsible for the development or production of different kinds of gum diseases.

### DETAILED DESCRIPTION OF THE DISCOVERY/INVENTION

Healing Rock is a trademark name that I selected for a kind of natural rock, which exists in the Mother Nature. I accidentally discovered that this rock contains some kind of natural remedy that is effective in healing the canker sores. This rock may be called with another names in another places for another usage. I chose to label this rock as Healing Rock only because of its feature for healing the canker sores that has never been tried before. This rock contains a type of medication not many have heard or nor ever used by many, but it amazingly works very well on canker sores. The natural ingredients contained in the Healing Rock have active fighting power of most processed medication, but do not present serious side effects like many prescribed and non-prescribed medication do (i.e. drowsiness, fatigue, nausea, pain, burns, and hurts). By having this type of medication on the market will provide many patients a natural alternative to curing their mouth canker sores. This is the reason why I am presenting this medication to all that suffer terrible and painful canker sores. It has lightweight crystallized structure with white color and sour taste. It is not edible. It is easily breakable by hand into small pieces. Healing Rock does not contain animal ingredients or has been tested on animals. There are no artificial colors, alcohol or other processed chemical found in Healing Rock. This is safe enough for the whole family to test out its natural remedy that can cure their mouth canker sores. Healing Rock is water-soluble rock that can be dissolved in hot water to form a fluid that is useful for gargling and rinsing the mouth not for drinking. It is not harmful if swallowed. Swallowing the fluid is not recommended.

What I claim is:

1- Discovery of a natural remedy contained inside the Healing Rock that is effective in healing the canker sores.

2- Invention of a natural oral mouthwash composition for treating canker sores.

3- Reserving a legal right for myself to be exclusive user of Healing Rock for medical purposes. Drug research laboratories, pharmaceutical manufacturing companies, or any other parties who are interested in using what I claimed in claim 1 and claim 2, must first obtain my permission before they can conduct further research, testing, development, production, sales, and marketing on Healing Rock to not only for canker sores, but also to other related gum diseases including gingivitis, bad breath, dental plaque, tooth decay, cold sores, warts, fever blisters, as well as the prevention, elimination, and/or reduction of oral microflora, bacteria, and/or other oral viral responsible for the development and/or production of canker sores and/or other related gum diseases.

1-1- The discovery as I claimed in claim 1, wherein said Healing Rock is only a trademark name that I selected for a kind of natural rock, which exists in the Mother Nature. This rock may be called with another names in another places for another usage. I chose to label this rock as Healing Rock only because of its feature for healing the canker sores that has never been tried before. Healing Rock is described in the following detail description section.

1-2- The discovery as I claimed in claim 1, wherein said natural remedy consists of pure natural ingredients derived from natural source concentrated inside the said Healing Rock that cure the canker sores without pain, side effects, burns, and/or hurts the sores like many other medication.

2-1- The invention as I claimed in claim 2, wherein said composition consists of a blend of some small pieces (about half ounce) of said Healing Rock as in claim 1, that are dissolved inside a cup of hot water. The chilled said composition is only for gargling and rinsing the mouth not for drinking or swallowing. The patients who suffer from the canker sores can rinse their mouth with the said composition for 30 seconds with two tablespoonfuls twice a day, early morning and late night for a few days until the content of the cup runs out. It's better off to brush the teeth just before rinsing the mouth with the said composition. It's also better off not to drink or eat for at least one hour after the rinsing the mouth with the said composition. Said composition has not been tried or tested on human bodies except on me. I have experienced the discomfort of canker sores for the past decade. I tried the said composition late November 2000, and it truly worked for me by actively curing my canker sores. Said composition is a fully natural and alcohol-free oral mouthwash fluid.
